(19) United States
(12) Patent Application Publication
(10) Pub. No.: US 2009/0112097 A1
(43) Pub. Date: Apr. 30, 2009

(54) ULTRASOUND IMAGING APPARATUS AND ULTRASOUND IMAGING METHOD

(76) Inventors: Sei Kato, Tokyo (JP); Hiroshi Hashimoto, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/255,381

(22) Filed: Oct. 21, 2008

(30) Foreign Application Priority Data
Oct. 24, 2007 (JP) 2007-276053

(51) Int. Cl. A61B 8/14

(52) U.S. Cl. 600/458

(57) ABSTRACT

An ultrasound imaging apparatus includes: an image acquisition unit for emitting ultrasound waves to a subject administered with a contrast agent and for obtaining a B-mode image of the subject; an image memory unit for storing a plurality of B-mode images, obtained by emitting ultrasound waves to the subject at a sound pressure destroying the contrast agent; and a combination image formation device for forming a combination image from a plurality of the B-mode images.

### CROSS REFERENCE TO RELATED APPLICATIONS

This application claims the benefit of Japanese Patent Application No. 2007-276053 filed Oct. 24, 2007, which is hereby incorporated by reference in its entirety.

### BACKGROUND OF THE INVENTION

The subject matter disclosed herein relates to an ultrasound imaging apparatus that observes an imaging area in a subject having a contrast agent administered by obtaining a B-mode image.

Recently, the contrast agent is imaged using an ultrasound imaging apparatus after administering the contrast agent to the subject to observe the contrast agent ingurgitated by for example Kupffer cells in the liver. This imaging requires the observation of the contrast agent distributed to a vast area in the liver.

As the contrast agent in this case acts as the heavy scatter material for the ultrasound waves, it yields the attenuation effect of the ultrasound waves so that the sound pressure behind the contrast agent will decrease. Thus for observing an organ such as the liver with the contrast agent distributed in a vast area by means of an ultrasound imaging apparatus, the contrast agent is observed clearly in the shallower area, while a significant decrease of sensitivity occurs in a deeper area due to the decrease of the ultrasound sound pressure.

In an ultrasound imaging method called flash method for destroying the contrast agent by emitting a high sound pressure ultrasound waves to the subject, a stronger signal is received which is generated when the ultrasound waves of the high sound pressure destroys the contrast agent, to display the distribution of the contrast agent as a tomographic image with higher sensitivity (see for example Japanese Unexamined Patent Publication No. 2005-074084).

When observing the tissue of the liver in which the contrast agent is distributed by using this flash, the contrast agent at the shallower area is imaged with higher sensitivity, then the attenuation effect at the shallower area is eliminated as the contrast agent in the shallower area is being destroyed, and sequentially the sound pressure of the ultrasound waves at the deeper area increases to destroy the contrast agent, and finally the distribution of the contrast agent will be imaged with higher sensitivity. The high sensitivity area indicating the distribution of the contrast agent in this manner moves in the direction from the shallower area to the deeper area as the time passes.

However, in accordance with the background art as have been described above, in an imaging area in which the contrast agent is distributed vastly from the shallower area to the deeper area, it may be difficult to grasp an entire distribution condition of the contrast agent with higher sensitivity. More specifically, when using the flash, the distribution condition of the contrast agent is observed with higher sensitivity, however, this is in a partial area in the scan direction of the ultrasound waves lying as a belt. In addition, the high brightness area of this belt shape moves in the direction from the shallower area to the deeper area within the short period of time of a few seconds. It is difficult to grasp the distribution condition in the entire area, of the contrast agent during this short period of time by the operator.

The formation of the imaging area that the contrast agent is vastly distributed as is in the tissue portion of the liver requires some time of five to ten minutes after the administration of the contrast agent to the subject. It is not preferable that the repetition of such a contrast agent examination for a plurality of times is a burden on the subject, as well as the efficiency of the examination.

From the reasons as have been described above, it is important to achieve an ultrasound imaging apparatus, which allows observing positively the systemic distribution of the contrast agent in the entire imaging area in which the contrast agent is vastly distributed from the shallower part to the deeper part, in an easier manner and by only one imaging action.

### BRIEF DESCRIPTION OF THE INVENTION

It is desirable that the problems described previously are solved.

An ultrasound imaging apparatus in accordance with first aspect of the invention includes: an image acquisition unit for emitting ultrasound waves to a subject administered with a contrast agent and for obtaining B-mode image information of the imaging area of the subject; an image memory unit for storing B-mode image information of a plurality of frames of the imaging area, obtained by emitting ultrasound waves to the subject at the sound pressure to destroy the contrast agent; and a combination image formation device using the plurality of frames of the B-mode image information to form one single combination image information of the imaging area.

In accordance with the invention in the first aspect, the combination image formation device uses a plurality of frames of the B-mode image information including the positional information of the high brightness area that changes along with the time, thereby to form one single item of the combination image information indicative of the entire distribution of the contrast agent.

An ultrasound imaging apparatus in accordance with second aspect of the invention, in the ultrasound imaging apparatus set forth in the first aspect described above, is characterized in that the combination image information is the maximum intensity projection image information by comparing the pixel values at the identical pixel position of the plurality of frames of the B-mode image information to set the maximum intensity among the pixel values as the pixel value of the pixel position.

The ultrasound imaging apparatus in accordance with third aspect of the invention, in the ultrasound imaging apparatus set forth in the first aspect described above, is characterized in that the B-mode image information includes the time information indicative of the time from the start of the emission until the acquisition of the frame, for each frame of the plurality of frames.

The ultrasound imaging apparatus in accordance with fourth aspect of the invention, in the ultrasound imaging apparatus set forth in the third aspect described above, is characterized in that the combination image formation device uses the time information to form the parameter value image information in which the parameter value calculated from the temporal change of the pixel values is set as the pixel value by using the time of starting the emission of the ultrasound waves at the destructive sound pressure as the starting time for each pixel constituting the B-mode image information.

The ultrasound imaging apparatus in accordance with fifth aspect of the invention, in the ultrasound imaging apparatus set forth in the fourth aspect described above, is characterized in that the parameter value is the peak pixel value that the pixel value is at the peak and the peak time that is from the start of the emission to the peak.

The ultrasound imaging apparatus in accordance with sixth aspect of the invention, in the ultrasound imaging apparatus set forth in the fifth aspect described above, is characterized in that the parameter value is the destruction termination time from the start of the emission to the decrease of the pixel value beyond the peak pixel value on the brightness change curve.

The ultrasound imaging apparatus in accordance with seventh aspect of the invention, in the ultrasound imaging apparatus set forth in the fifth aspect described above, is characterized in that the parameter value is the destruction continuation time which is the difference between the destruction termination time and the peak time.

The ultrasound imaging apparatus in accordance with eighth aspect of the invention, in the ultrasound imaging apparatus set forth in any one of the first to seventh aspects described above, is characterized in that the ultrasound imaging apparatus includes a display unit for color displaying the combination image information, and an image display controller unit for controlling the color display.

In the eighth aspect of the invention, the color display of the combination image information is performed in the ultrasound imaging apparatus.

The ultrasound imaging apparatus in accordance with ninth aspect of the invention, in the ultrasound imaging apparatus set forth in any one of the first to eighth aspects described above, is characterized in that the combination image formation device includes a hue association device for associating the pixel value of the combination image information to the hue.

The ultrasound imaging apparatus in accordance with tenth aspect of the invention, in the ultrasound imaging apparatus set forth in the ninth aspect described above, is characterized in that the ultrasound imaging apparatus includes an image display controller unit for color displaying the combination image information associated to the hue.

The ultrasound imaging apparatus in accordance with eleventh aspect of the invention, in the ultrasound imaging apparatus set forth in any one of the first to tenth aspects described above, is characterized in that the ultrasound imaging apparatus includes an input unit for setting a plurality of ROIs each having a different depth in the depth direction of the emission of ultrasound waves in the imaging area.

The ultrasound imaging apparatus in accordance with twelfth aspect of the invention, in the ultrasound imaging apparatus set forth in the eleventh aspect described above, is characterized in that the ultrasound imaging apparatus includes a representative pixel value calculation device, which uses the pixel value of the B-mode image information for each of the ROIs to calculate a maximum intensity of the pixel value within the ROIs, an addition value of the pixel value within the ROIs, or an addition mean value of the pixel values within the ROIs as the representative pixel value.

The ultrasound imaging apparatus in accordance with thirteenth aspect of the invention, in the ultrasound imaging apparatus set forth in the twelfth aspect described above, is characterized in that the ultrasound imaging apparatus includes a ROI detection device, which uses the representative pixel value to detect the ROI which is the highest brightness by the emission of the ultrasound waves of the destructive sound pressure.

The ultrasound imaging apparatus in accordance with fourteenth aspect of the invention, in the ultrasound imaging apparatus set forth in the thirteenth aspect described above, is characterized in that the ultrasound imaging apparatus includes a focal point controller device for changing the location of the focal point of the ultrasound waves based on the detection.

The ultrasound imaging apparatus in accordance with fifteenth aspect of the invention, in the ultrasound imaging apparatus set forth in the fourteenth aspect described above, is characterized in that the ultrasound imaging apparatus includes a frequency controller device for changing the excitation frequency of the ultrasound waves based on the detection.

In the fifteenth aspect of the invention, the excitation frequency of the ultrasound waves is set to be different for each ROI.

In accordance with the invention, the systemic state of distribution of the contrast agent distributing from the shallower area to the deeper area of the imaging area is easily obtained from one single combination image information obtained by one imaging action, and more specifically the distribution of the contrast agent which varies in the direction of depth can be clearly recognized by the comparison of the distribution in one single combination image information.

Further objects and advantages of the present invention will be apparent from the following description of the preferred embodiments of the invention as illustrated in the accompanying drawings.

### BRIEF DESCRIPTION OF THE DRAWINGS

FIG. 1 shows a schematic block diagram illustrating the entire configuration of the ultrasound imaging apparatus;

FIG. 2 shows a schematic block diagram illustrating the configuration of a controller unit in accordance with a first embodiment;

FIG. 3 shows a flow chart illustrating the operation of the ultrasound imaging apparatus in accordance with the first embodiment;

FIG. 4 shows a flow chart illustrating the operation of the partial high brightness image acquisition processing in accordance with the first embodiment;

FIG. 5 shows a schematic diagram illustrating a B-mode image obtained in the partial high brightness image acquisition processing;

FIG. 6 shows a flow chart illustrating the operation of the combination image formation processing in accordance with a second embodiment;

FIG. 7 shows a schematic diagram illustrating an MIP image formed by the combination image formation means;

FIGS. 8(A) through 8(D) show schematic diagrams illustrating the brightness change curve for each pixel to be calculated by the combination image formation means;

FIG. 9 shows a schematic diagram illustrating the parameter values to be calculated from the brightness change curve;

FIG. 10 shows a schematic diagram illustrating an example of the lookup table associating the pixel value with the hue;

FIGS. 11(A) and 11(B) show schematic diagrams illustrating the color image information to be formed in the combination image formation means;

FIG. 12 shows a schematic block diagram illustrating the configuration of the controller unit in accordance with a second embodiment;

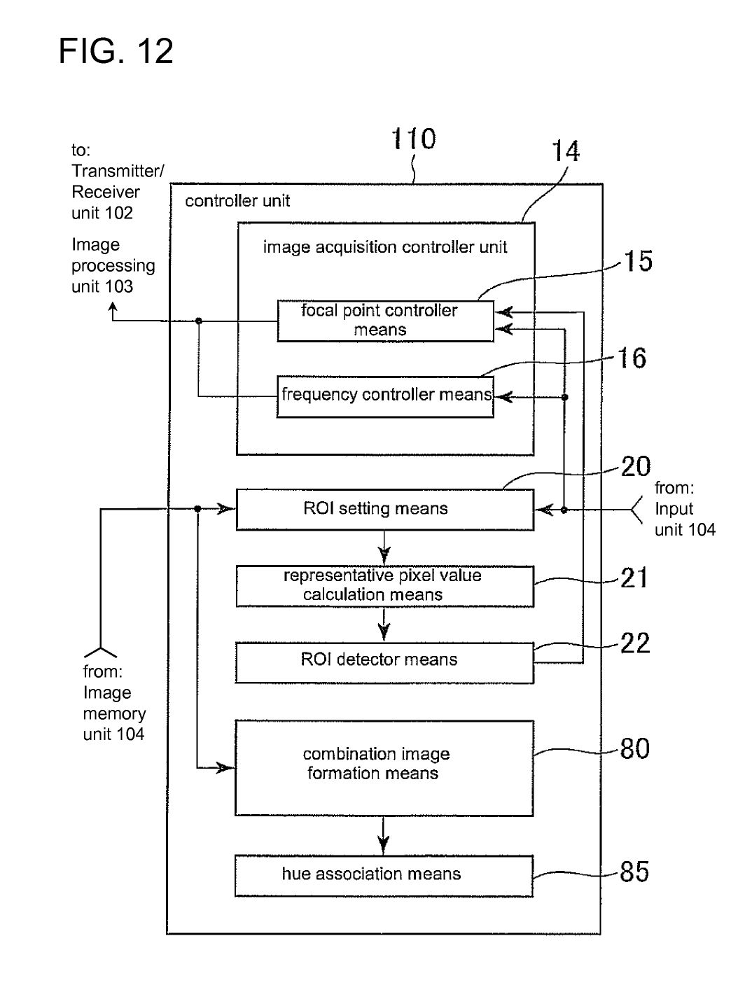

FIG. 13 shows a flow chart illustrating the operation in the partial high brightness image acquisition processing in accordance with the second embodiment;

FIG. 14 shows a schematic diagram illustrating an example of ROI to be set to the imaging area in accordance with the second embodiment; and

FIG. 15 shows a schematic diagram illustrating how to determine the change in time course of the representative pixel value of the ROI as well as the time that the representative pixel value is beyond the peak.

### DETAILED DESCRIPTION OF THE INVENTION

Various embodiments of the invention for the ultrasound imaging apparatus will be described in greater details with reference to the accompanying drawings. It should be noted here that the embodiments described herein are not considered to limit the invention.

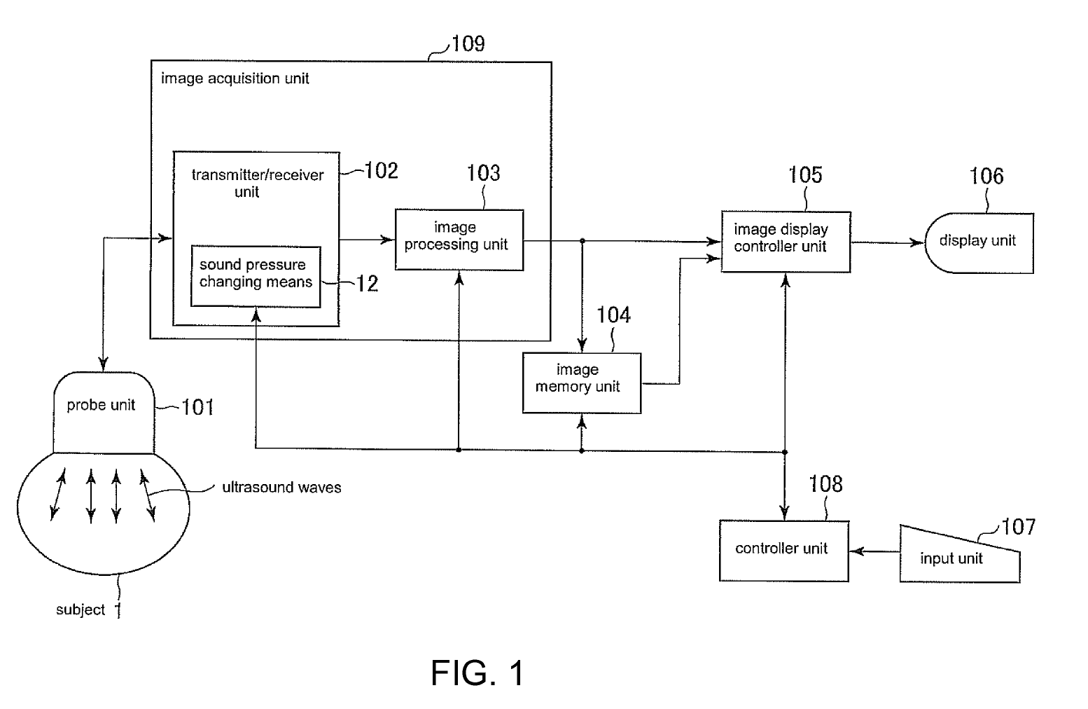

The entire configuration of the ultrasound imaging apparatus in accordance with a first embodiment of the invention will be described now. FIG. 1 shows a schematic block diagram illustrating the entire configuration of the ultrasound imaging apparatus in accordance with the first embodiment. The ultrasound imaging apparatus includes a probe unit 101, an image acquisition unit 109, an image memory unit 104, an image display controller unit 105, a display unit 106, an input unit 107, and a controller unit 108. The image acquisition unit 109 further includes a transmitter/receiver unit 102 and an image processing unit 103.

The probe unit 101 is the part for transmitting and receiving the ultrasound waves, in other words it is the part for iteratively emitting the ultrasound waves in a specific direction to the imaging section of a subject 1 and for receiving the ultrasound signals reflected from the inside of the subject 1 as the sound lines of time series. The probe unit 101 also performs the electronic scan by sequentially switching the emitting direction of the ultrasound waves. Although not shown in the figure, the probe unit 101 has piezoelectric elements arranged as an array.

The transmitter/receiver unit 102 is connected to the probe unit 101 via a concentric cable, for performing the primary stage amplification of the electric signal for driving the piezoelectric elements in the probe unit 101 and of the received ultrasound signals. The transmitter/receiver unit 102 also includes a sound pressure changing means 12. The sound pressure changing means 12 is responsive to the control signals from the controller unit 108 to change the voltage for driving the piezoelectric elements. The sound pressure changing means 12 will set the sound pressure of the ultrasound waves to emit to the subject to the destructive sound pressure for destroying the contrast agent administered to the subject.

The image processing unit 103 performs the formation of electric signals for driving the transmitter/receiver unit 102, and the formation of tomographic image information from the ultrasound signals amplified by the transmitter/receiver unit 102. In particular when the contrast agent is administered to the subject 1, it performs a contrast mode processing for generating the contrast mode images in real-time basis.

The image processing unit 103 performs, for example as specific processing contents, delays the transmission signals to focus at the focal point, in the case of transmission of ultrasound waves. In the case of reception of ultrasound waves it performs a delay addition processing of the received ultrasound waves signals, an analog-to-digital conversion processing, a writing processing for writing thus converted digital information into the image memory unit 104, which will be described below, as the B-mode image information.

The image memory unit 104 is an image memory for storing the B-mode image information generated by the contrast mode processing. More specifically, the image memory unit 104 stores, together with the acquisition time information of imaging, the B-mode image information that varies along with the time with a frame that constitutes one single item of the tomographic image information of the imaging area as a minimum unit.

The image display controller unit 105 performs a display frame rate conversion of the B-mode image information generated by the image processing unit 103, a color display control, as well as the control of shape and position of the display image of the B-mode image information. It also displays the ROI (region of interest) indicative of the region of interest on the display image of the B-mode image information.

The display unit 106 uses such a display as CRT (cathode ray tube) or an LCD (liquid crystal display) to visually display the image information output from the image display controller unit 105 to the operator. The display unit 106 is also capable of color displaying in correspondence with the instruction from the image display controller unit 105.

The input unit 107 is constituted of a keyboard and a pointing device, for transferring to the controller unit 108 the operation instruction signals input by the operator for setting the emission ultrasound waves to be the destructive sound pressure, and the operational input signals for selecting whether or not to display by the B-mode image. The input unit 107 performs, on the tomographic image displayed on the display unit 106, the position setting for setting the region of interest such as ROIs and the decision input of the position.

The controller unit 108 controls the operation of the components of the ultrasound imaging apparatus as have been described above based on the operation input signal provided from the input unit 107 and the program and data previously stored, and displays the B-mode image on the display unit 106.

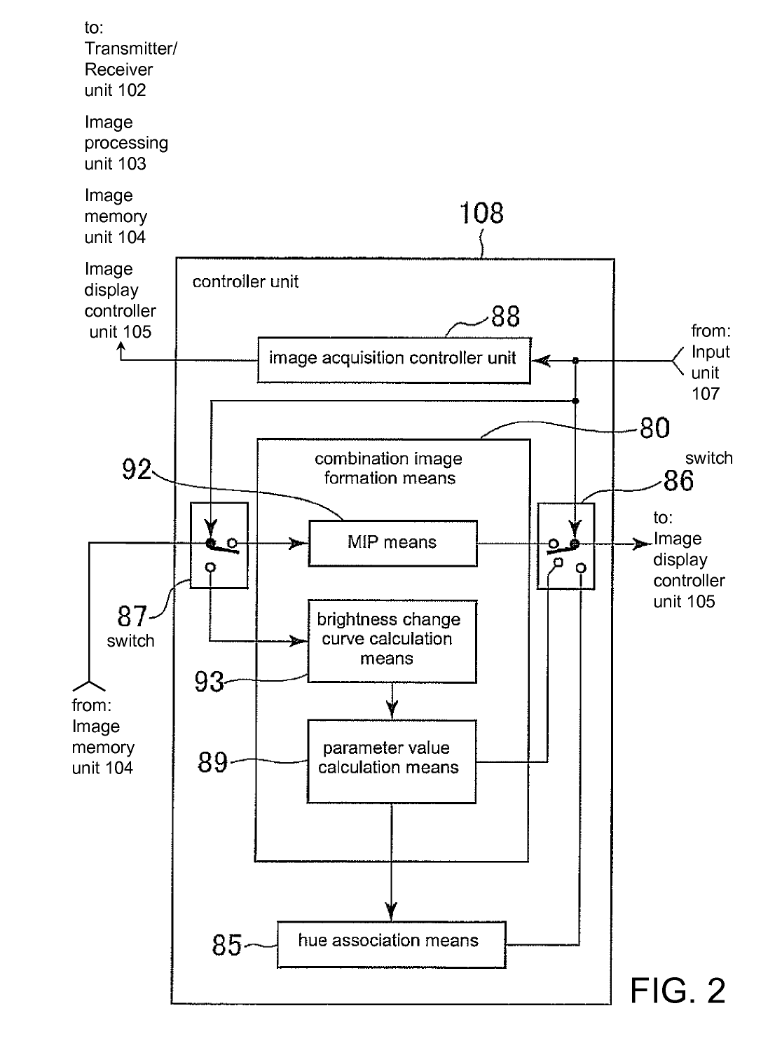

Now referring to FIG. 2 there is shown a schematic block diagram illustrating the configuration of the controller unit 108. The controller unit 108 includes an image acquisition controller unit 88, a combination image formation means 80, a hue association means 85, switches 86 and 87. The combination image formation means 80 includes an MIP (maximum intensity projection) means 92, a brightness change curve calculation means 93, and a parameter value calculation means 89.

The image acquisition controller unit 88 performs an ultrasound scan based on the scan information including the imaging mode specification information, the driving voltage information, the focal point information, the driving frequency information and the like from the input unit 107 to obtain the tomographic image information. More specifically, the image acquisition controller unit 88 uses the sound pressure changing means 12 by the designation by the input unit 107 to set the ultrasound waves pulses to be emitted to be the destructive sound pressure, and to store the B-mode image information to be obtained into the image memory unit 104 until instructed to stop by the input unit 107 while emitting the ultrasound waves of the destructive sound pressure to the subject.

The combination image formation means 80 forms the combination image information indicative of the distribution of the contrast agent in the entire area of the imaging area based on a plurality of frames of the B-mode image information stored in the image memory unit 104 and in the case of using the destructive sound pressure for the contrast agent as the ultrasound waves. The pixel values in the combination image information are determined based on the pixel values of the B-mode image information stored in the image memory unit 104.

The MIP means 92 determines the maximum intensity projection image information from a plurality of frames of the B-mode image information stored in the image memory unit 104. The MIP means 92 compares the pixel values positioned at the same pixel position in a plurality of frames of the B-mode image information to determine the maximum pixel value from within these pixel values, then it determines a new maximum intensity projection image information that uses the maximum pixel value as the pixel value at the same image pixel position.

The brightness change curve calculation means 93 uses the time information associated to each frame to determine the brightness change curve indicative of the change of pixel values along with the time for each pixel position of the B-mode image information, from a plurality of frames of the B-mode image information stored in the image memory unit 104.

The parameter value calculation means 89 uses the brightness change curve of each pixel position determined by the brightness change curve calculation means 93 to determine the parameter values such as the peak time and so on. Then it determines the parameter value image information using these parameter values as the pixel values.

A specific example of the maximum intensity projection image information, the brightness change curve, and the parameter value image information as have been described above will be described in greater details later along with the operation of the controller unit 108.

The switches 86 and 87 select the image formation performed by the combination image formation means 80 and the display of thus formed combination image information, in accordance with the instruction from the input unit 107 The hue association means 85 will be selected when color displaying the combination image information.

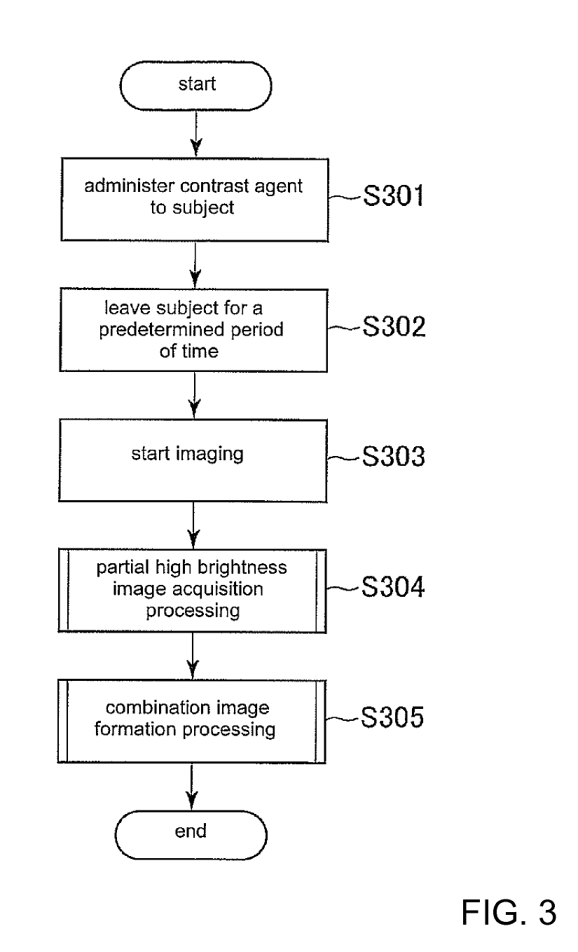

Next, the flow charts shown in FIG. 3 to FIG. 5 will be used to describe the operation of the controller unit 108. The operator first administers the contrast agent via a vein of the subject 1 (step S301). The contrast agent is composed of bubbles having the diameter of a few μm, which generates strong signals by destroying the shell of the bubbles by emitting the ultrasound waves of high sound pressure.

Thereafter, the subject 1 is instructed to stay still for a predetermined period of time (step S302). The stay time is approximately five to ten minutes, during which time the contrast agent administered through the vein may pass through the heart to circulate in the body. Then, the contrast agent flowing into the liver may be ingurgitate by the Kupffer cells of the tissue through the capillaries. The distribution of the density of the contrast agent absorbed into the tissue may almost reflect the distribution of the density of Kupffer cells.

Thereafter, the operator starts to image (step S303), and place the probe unit 101 in contact with the objective imaging area of the subject 1, for example, such as the imaging section position of the liver, while imaging in B-mode.

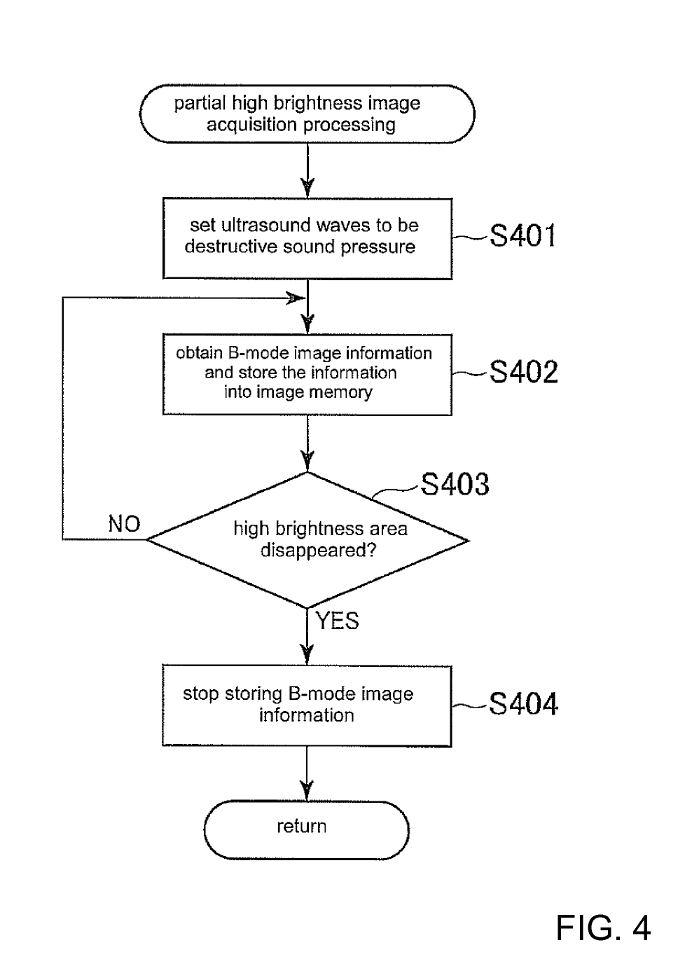

Then the operator performs the partial high brightness image acquisition processing of the contrast agent (step S304). FIG. 4 shows a flow chart illustrating the operation of the partial high brightness image acquisition processing. The operation instructs to start the operation of the partial high brightness image acquisition processing by using a keyboard from the input unit 107. In the partial high brightness image acquisition processing the sound pressure of the ultrasound waves emitted to the subject 1 from the probe unit 10 is set to the destructive sound pressure by the sound pressure changing means 12 (step S401).

Then the partial high brightness image acquisition processing obtains a plurality of frames of the B-mode image information while holding the sound pressure of the ultrasound waves to be the destructive sound pressure, and stores them to the image memory unit 104 (step S402).

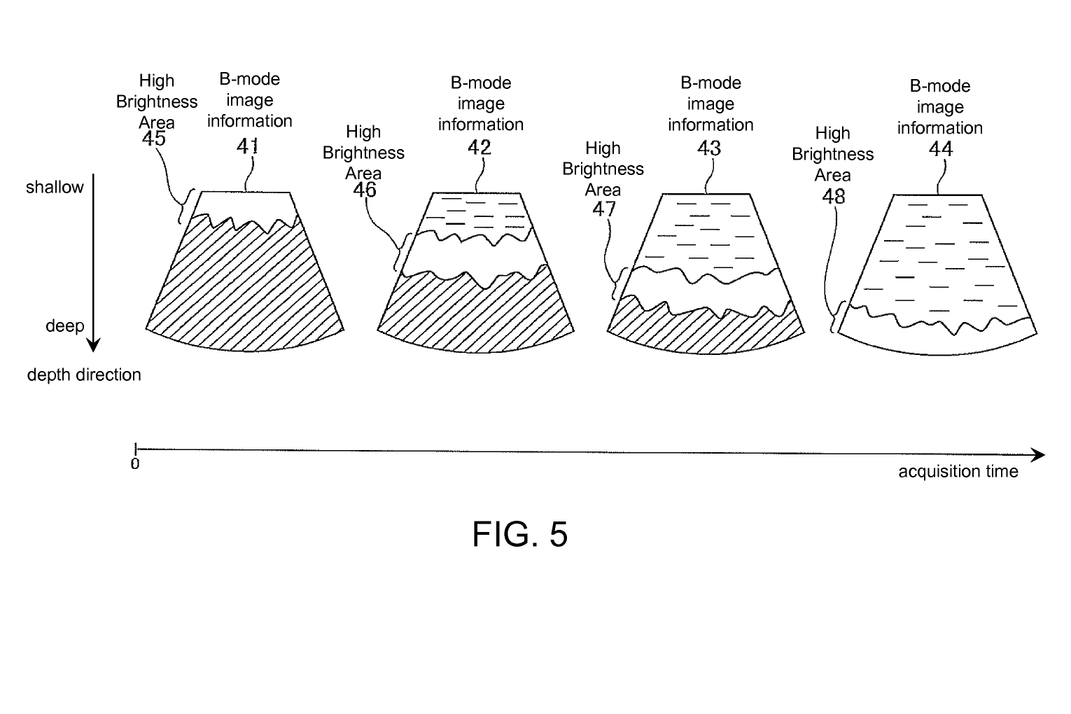

Now referring to FIG. 5, there is shown a schematic diagram illustrating a plurality of frames of the B-mode image information stored in the image memory unit 104 in synchronism with the time axis from the time of the start of acquisition. The axis of abscissas is the time axis indicating the acquisition time of the B-mode image information, using as the start time the time of setting the sound pressure of the ultrasound waves to be the destructive sound pressure. The B-mode image information 41 to 44 is arranged at the positions of the acquisition time along with the time axis, and shown in the figure such that the direction perpendicular to the acquisition time is the direction of depth of the B-mode images. The imaging area of the B-mode image information 41 to 44 is assumed that the contrast agent is almost uniquely distributed.

The B-mode image information 41 is a B-mode image obtained immediately after setting the sound pressure of the ultrasound waves to be the destructive sound pressure. The shallower area in the depth direction of the B-mode image information 41 depicts a high brightness area 45 caused by the signals generated by the destroyed contrast agent. On the other hand, in the imaging area in the depth direction deeper than the high brightness area 45, the sound pressure will be decreased by the reflection of the ultrasound waves caused by the contrast agent in the high brightness area 45. Therefore the imaging area in the depth direction deeper than the high brightness area 45 as shown by the oblique lines in the figure will be the image having less effect of the contrast agent.

The B-mode image information 42 is a B-mode image to be obtained after the contrast agent present in the high brightness area 45 of the shallower area is destroyed. The B-mode image 42 has another new high brightness area 46 at the position adjacent to the high brightness area 45 in the depth direction. In the high brightness area 46 there is the contrast agent not yet destroyed, and the signal emitted at the time when the contrast agent is destroyed forms the high brightness area 46. In the direction shallower than the high brightness area 46 a B-mode image is generated while in the direction deeper than the high brightness area 46 a B-mode image has less effect of the contrast agent.

The B-mode image information 43 is a B-mode image to be acquired after the contrast agent present in the high brightness area 46 has been destroyed. The B-mode image 43 has still another high brightness area 47 at the position adjacent to the high brightness area 46 in the depth direction. There is the contrast agent not yet destroyed present in the high brightness area 47, and the high brightness area 47 will be formed by the signals emitted at the time when the contrast agent is destroyed.

The B-mode image information 44 is a B-mode image to be acquired after the contrast agent present in the high brightness area 47 has been destroyed. The B-mode image information 44 has still another new high brightness area 48 at the position adjacent to the high brightness area 47 in the depth direction. In the high brightness area 48 there is present the contrast agent not yet destroyed, and the high brightness area 48 will be formed by the signals emitted at the time when the contrast agent is destroyed. The high brightness area 48 is the deepest area on the B-mode image information 44 in the depth direction.

In the partial high brightness image acquisition processing, as is shown in the B-mode image information 41 to 44, B-mode images are acquired with the high brightness areas 45 to 48 moving from the shallower to deeper area, along with the elapsed time of acquisition. The width of the high brightness areas 45 to 48 in the depth direction will be different by the density of the contrast agent present in the imaging area as well as the sound pressure of the ultrasound waves. There are needed approximately a few seconds from the time when setting the sound pressure of the ultrasound waves to be the destructive sound pressure to the time when the high brightness area reaches to the deepest area of the imaging area, and the number of B-mode image information actually acquired during this period of time will be approximately in the order of several tens of frames. FIG. 5 shows the time course of the high brightness area simplified with less number of frames for the sake of clarity.

Thereafter, returning to FIG. 4, the operator will determine whether or not a high brightness area is disappeared from the B-mode image on the display unit 106 (step S403). The operator proceeds to step S402 if on the B-mode image there is a high brightness area (step S403 negative) and instructs to repeat the acquisition of the B-mode image information and the storage of them into the image memory unit 104. If the high brightness areas are disappeared from the B-mode image (step S403 positive), the operator then instructs to decrease the sound pressure of the transmitted ultrasound waves and to stop storing the B-mode image information into the image memory unit, from the input unit 107 (step S404), the process proceeds back to the main routine after stopping the partial high brightness image acquisition processing.

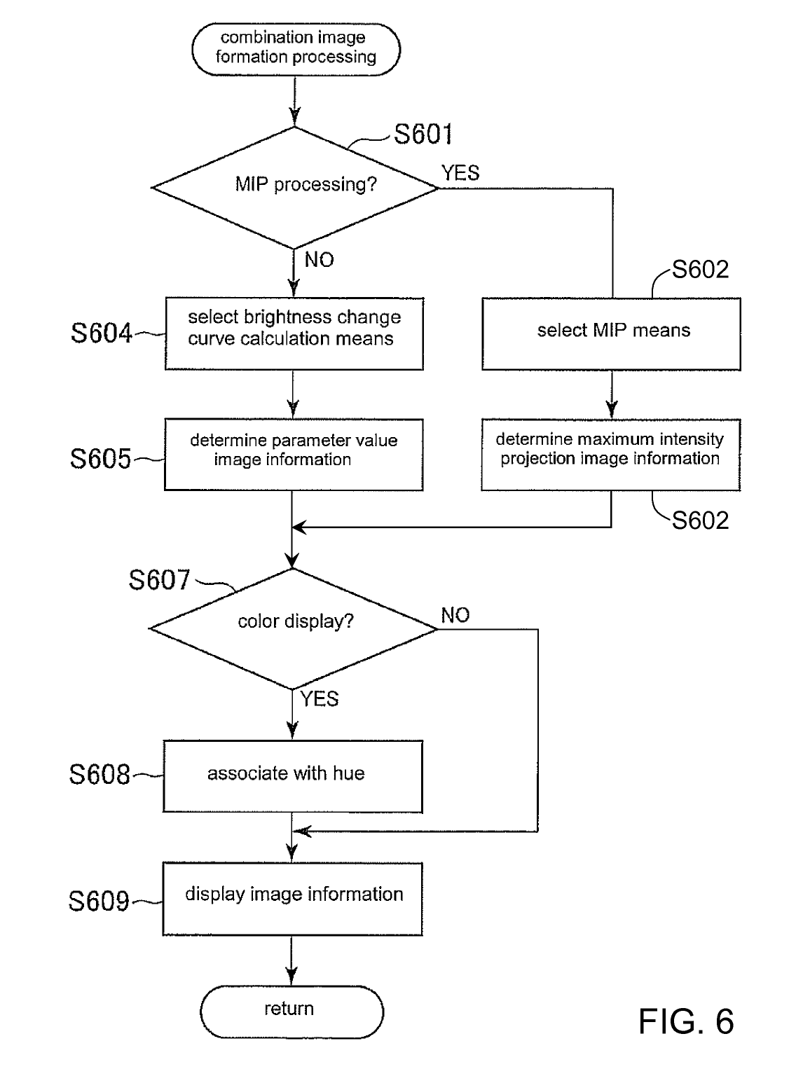

Then, returning to FIG. 3, the operator instructs to perform a combination image formation processing (step S305). FIG. 6 shows a flow chart illustrating the operation of the combination image formation processing. First the operator determines whether or not an MIP processing is to be applied to the B-mode image information 41 to 44 as shown in FIG. 5, which are stored in the image memory unit 104 (step S601). Then, if the MIP processing is applied (step S601 positive), the operator uses the switches 86 and 87 to select the MIP means 92 from the input unit 107 (step S602) to determine the maximum intensity projection image information which is the combination image information from the B-mode image information 41 to 44 stored in the image memory unit 104 (step S603).

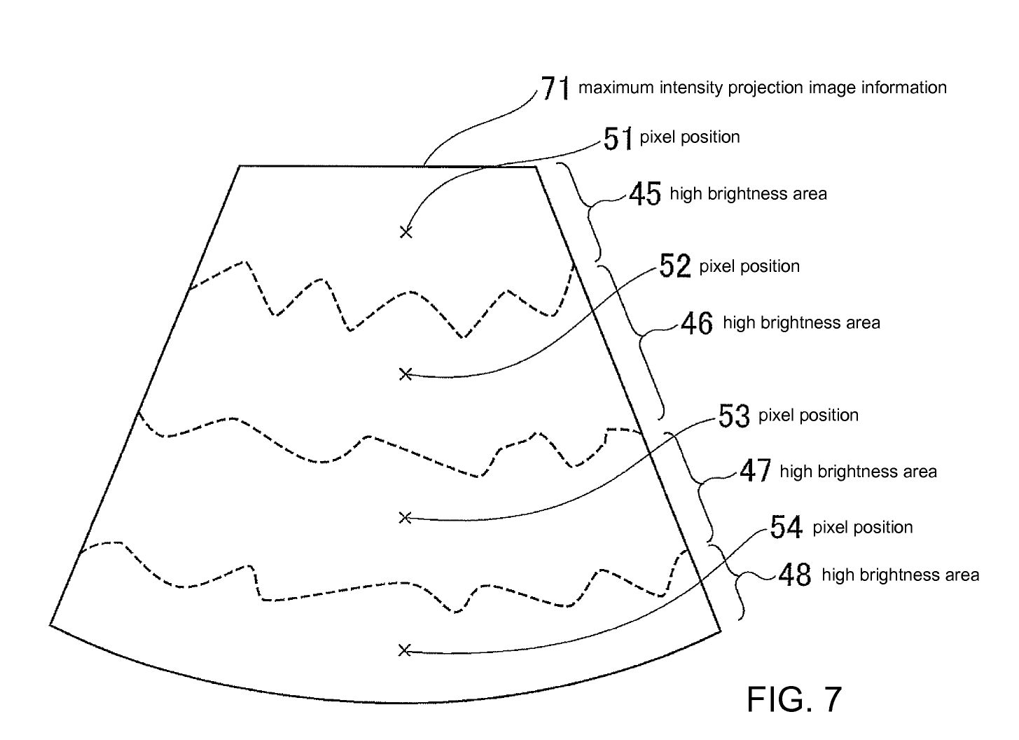

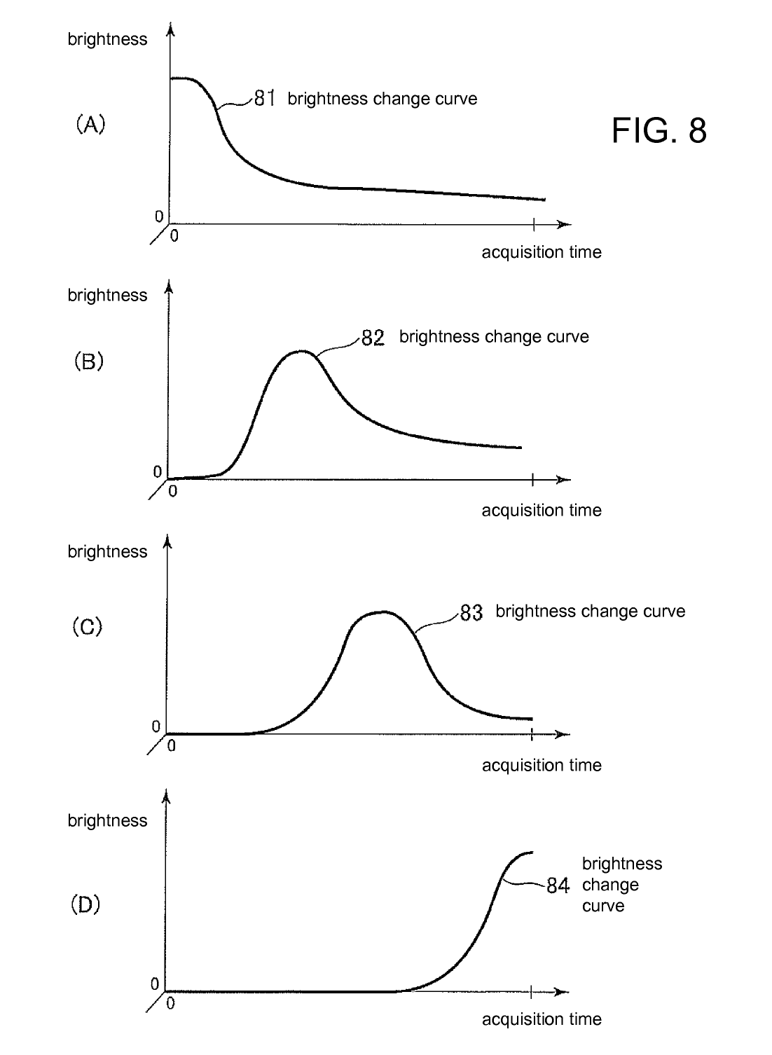

Now referring to FIG. 7 there is shown a schematic diagram illustrating the maximum intensity projection image information 71 given by using the B-mode image information 41 to 44. The MIP means 92 determines the maximum intensity among the pixel values (brightness values) at the same pixel position of the B-mode image information 41 to 44, and then forms maximum intensity projection image information 71 with this maximum intensity as the new pixel value. Therefore, from the B-mode image information 41 to 44, which have the high brightness areas 45 to 48 caused by the destruction of the contrast agent, maximum intensity projection image information 71 is formed which is one single combination image information that combines the high brightness areas 45 to 48, by the MIP processing. The dotted line in the figure illustrates the border between the high brightness areas 45 to 48. The high brightness areas 45 to 48 may have the nonuniformity of brightness not shown in the figure, the nonuniformity of brightness may reflect the distribution of the density of the contrast agent present in the imaging area. The pixel positions 51 to 54, shown within each high brightness area 45 to 48 included in the maximum intensity projection image information 71 of FIG. 7, will be described in greater details later with reference to FIG. 8.

Then returning to FIG. 6, if the MIP processing is not applied (step S601 negative), the operator uses the switches 86 and 87 to select the brightness change curve calculation means 93 from the input unit 107 (step S604), to instruct to calculate the brightness change curve. The brightness change curve is a curve indicative of the change along with the time of the brightness for each of the same pixel positions in the B-mode image information 41 to 44 which are the images of the same imaging area.

FIGS. 8(A) through 8(D) show examples of the brightness change curve at some typical pixel positions from a shallower area to a deeper area. For these pixel positions, the pixel positions 51 to 54 may be used which are shown in the high brightness area 45 to 48 shown in FIG. 7, and the brightness change curves at the pixel positions 51 to 54 are shown as FIG. 8(A) to FIG. 8(D).

FIG. 8(B) and FIG. 8(C) illustrate exemplary brightness change curves 82 and 83, which are indicated by the pixels of the high brightness areas 46 and 47 present in the imaging area of the B-mode image information 41 to 44 and positioned at the intermediate depth area in the depth direction. Since the contrast agent is present in the shallower area immediately after starting imaging, the brightness change curves 82 and 83 have less effect of the contrast agent and lower brightness in the deeper area. On the other hand some time after starting imaging, the contrast agent in the shallower area will have been destroyed and the sound pressure in the intermediate area will increase, and the contrast agent present in the intermediate area will form the high brightness areas 46 and 47, so that a peak will be appeared in the brightness change curve. Thereafter, since there is not the contrast agent in the shallower and intermediate areas, lower brightness will be appeared in the brightness change curves 82 and 83 indicating the reflected ultrasound waves echoes from the tissue of the intermediate area.

FIG. 8(B) and FIG. 8(C) illustrate exemplary brightness change curves 82 and 83, which are indicated by the pixels of the high brightness areas 46 and 47 present in the imaging area of the B-mode image information 41 to 44 and positioned at the intermediate depth area in the depth direction. Since the contrast agent is present in the shallower area immediately after starting imaging, the brightness change curves 82 and 83 have less effect of the contrast agent and lower brightness in the deeper area. On the other hand some time after starting imaging, the contrast agent in the shallower area will have been destroyed and the sound pressure in the intermediate area will increase, and the contrast agent present in the intermediate area will form the high brightness areas 46 and 47, so that a peak will be appeared in the brightness change curve. Thereafter, since there is not the contrast agent in the shallower and intermediate areas, lower brightness will be appeared in the brightness change curves 82 and 83 indicating the reflected ultrasound waves echoes from the tissue of the intermediate area.

FIG. 8(D) indicates an exemplary brightness change curve 84, which is indicated by the pixel in the high brightness area 48 present at the end in the depth direction in the imaging area of the B-mode image information 41 to 44. For some time after starting imaging, there is present the contrast agent in the shallower area and in the intermediate area, and on the brightness change curve 84 there is less effect of the contrast agent in the deeper area so that the brightness will be lower. When the contrast agent present in the shallower area and in the intermediate area is destroyed and that the sound pressure is increasing in the deeper area, the contrast agent present in the deeper area will form the high brightness area 48 and a peak will be appeared in the brightness change curve 84.

Thereafter, returning to FIG. 6, the operator uses the parameter values calculated from the brightness change curve based on the brightness change curve determined for each pixel position, to determine the parameter value image information, which is the combination image information (step S605). The parameter value is specified from the input unit 107 by the operator, which value reflects the distribution of the contrast agent present in the tissue.

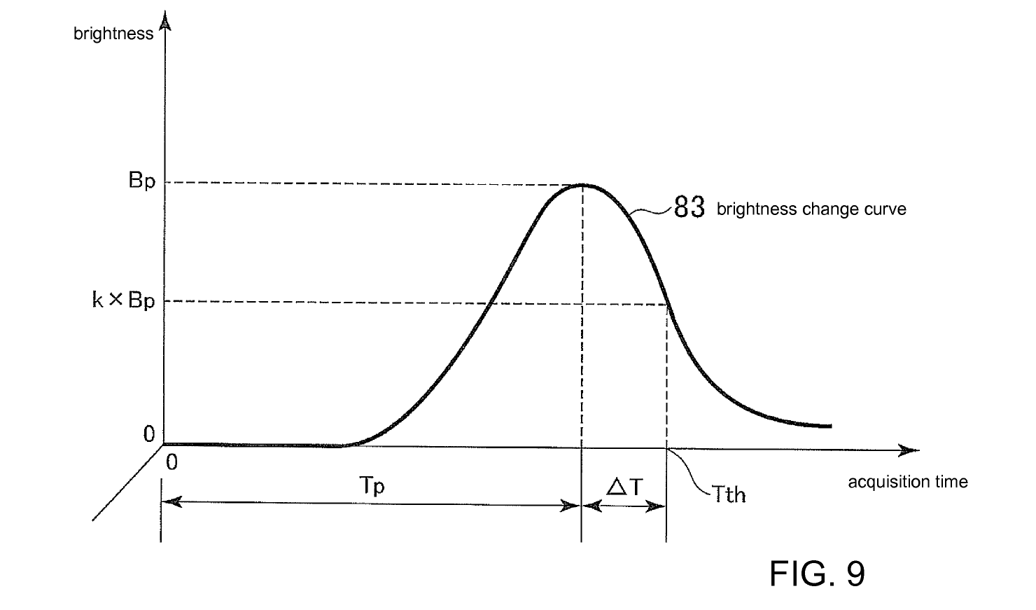

FIG. 9 shows a schematic diagram illustrating an example of parameter value determined based on the brightness change curve. In FIG. 9 there is shown a brightness change curve 83 of the pixel positioned in the intermediate area in the depth direction as an example. The brightness change curve 83 has a peak at the point of time where the contrast agent that is present in some part of the shallower area and the intermediate area is destroyed. Around this peak many parameter values of the brightness change curve 83 will be given. Some parameter values of the brightness change curve 83 include the peak value Bp, the peak time Tp, the destruction termination time Tth, the destruction continuation time Δ T, and the like.

The peak value Bp is a peak value that the peak of the brightness change curve 83 has, which is the value approximately proportional to the density of the contrast agent at this pixel position. The peak time Tp is the period of time from when the sound pressure of the ultrasound waves is set to the destructive sound pressure until the contrast agent at this pixel position is almost destroyed, which depends on the pixel position in the depth direction, and on the distribution of the density of the contrast agent up to the depth position. The destruction termination time Tth is the time from when the sound pressure of the ultrasound waves is set to the destructive sound pressure until the destruction of the contrast agent at this pixel position is almost terminated, which will be the amount that reflects the peak width of the brightness change curve 83. The destruction termination time Tth is the acquisition time which has the longer period of time between two acquisition times at the brightness value Bp×k where the value decreases by the coefficient k (<1) from the peak value Bp in the brightness change curve 83. The destruction continuation time Δ T is the value that the peak time Tp is subtracted from the destruction termination time Tth, which is the amount that reflects the time required for destroying the contrast agent at this pixel position. These parameter values are calculated which are selected from the input unit 107 by the operator, and in correspondence with the selected parameter values, the parameter value image information that has the peak value Bp as the pixel value, the parameter value image information that has the peak time Tp as the pixel value, the parameter value image information that has the destruction termination time Tth as the pixel value, and the parameter value image information that has the destruction continuation time Δ T as the pixel value will be formed.

When the operator selects the peak time Tp as the parameter value, the image will be that the brightness increases as the depth is deeper in the depth direction.

In the foregoing description, for the sake of clarity, these parameter values are determined from the brightness change curve. However, by tracking the change in the time course of the brightness, these parameter values may be given during the tracking, without determining the brightness change curve.

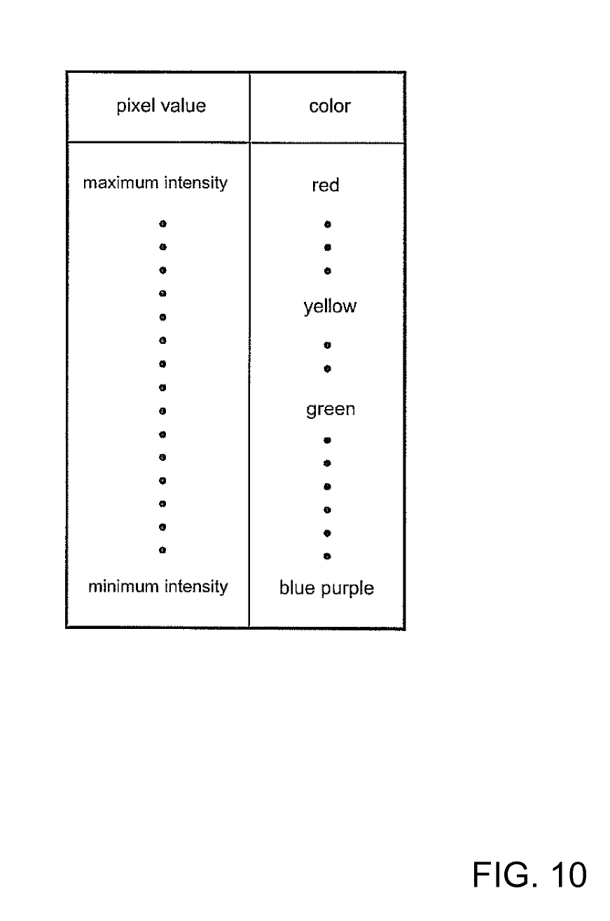

Thereafter, returning to FIG. 6, the operator will determine whether or not the MIP image information or the parameter value image information will be color displayed (step S607). In case not to color display the MIP image information or the parameter value image information (step S607 negative), the operator instructs to display the image information having the pixel value of the MIP image information or of the parameter value image information as the brightness information (step S609). In case of color displaying the MIP image information or the parameter value image information (step S607 positive), the hue association means 85 is invoked to associate the pixel value in the MIP image information or in the parameter value image information with the hue (step S608). The hue association means 85 uses for example a lookup table as shown in FIG. 10 to associate the pixel value in the MIP image information or in the parameter value image information with the hue. For the lookup table, as an example, a table is shown in which the pixel value from the minimum intensity to the maximum intensity is associated to the visual light from blue purple to red. The hue is specified by using the code such as RGB expression, which is matched with the hue code to instruct to the display unit 106 by the image display controller unit 105.

Then, the controller unit 108 displays on the display unit 106 the MIP image information or the parameter value image information having the color display code as the pixel value (step S609), then it terminates this combination image formation processing and the main routine shown in FIG. 3.

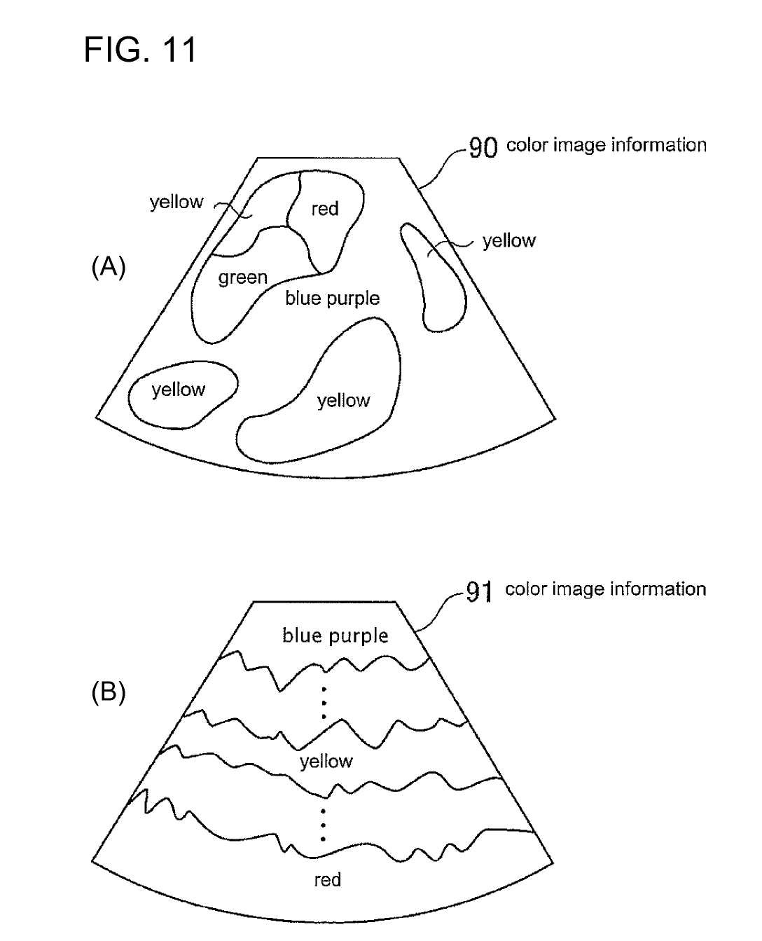

FIGS. 11(A) and 11(B) show schematic diagrams illustrating the color image information to be displayed on the display unit 106. FIG. 11(A) is an example of the color image information 90 to be displayed on the display unit 106. The contrast agent is distributed in the entire area of the imaging area, and the color image information 90 has a color reaching to red if the contrast agent is distributed at a higher density, and has a color reaching to blue purple if the contrast agent is distributed at a lower density. The color image information 90 indicates the color displayed distribution of the density of the contrast agent in the entire section from the shallower position to the deeper position in the depth direction.

FIG. 11(B) is a schematic diagram illustrating a color display of the parameter value image information that uses the peak time Tp as the parameter value. As the peak time Tp is, roughly, shorter at the shallower position in the depth direction, and longer at the deeper position in the depth direction, the color image information 91 changes color approximately from blue purple to red along with the transition of position from a shallower position to a deeper position, so as to indicate as a color display the change in the distribution of the contrast agent in the depth direction.

As have been described above, in the first embodiment, while emitting the ultrasound having the sound pressure for destroying the contrast agent to the subject 1, a plurality of frames of the B-mode image information 41 to 44 is acquired, and from the image information, the maximum intensity projection image information or the parameter value image information which may be the combination image information is formed and displayed. The information on the density distribution of the contrast agent in the entire area in the imaging area may be acquired by one single imaging session at once, and then the information on the density distribution in the imaging area may be easily caught as one single combination image information.

In this first embodiment, in step S403, although the operation visually determines whether or not the high brightness area is disappeared from the B-mode image on the display unit 106, this step may be automated. When performing automatically, for example a threshold value is provided to the pixel value of the B-mode image, and if the pixel value is beyond the threshold value, then there will be a high brightness area, while if the pixel value is less than the threshold value, then the high brightness area is assumed to be disappeared.

Although in the first embodiment described above, the imaging conditions such as the focal point of the ultrasound waves in the depth direction when acquiring the B-mode image information is fixed, in the partial high brightness image acquisition processing for acquiring a plurality of frames of the B-mode image information by setting the sound pressure of the ultrasound waves to be the destructive sound pressure, these imaging conditions may be optimized for the acquired B-mode image information to allow forming a much clearer combination image information. In a second embodiment of the invention there will be illustrated a case in which the imaging conditions are optimized in accordance with the acquired B-mode image information.

The ultrasound imaging apparatus to be used in the second embodiment includes a controller unit 110 instead of the controller unit 108 shown in FIG. 1. Other components of the ultrasound imaging apparatus are identical to that shown in FIG. 1 and the detailed description thereof will be omitted.

The controller unit 110 includes an image acquisition controller unit 14, an ROI setting means 20, a representative pixel value calculation means 21, an ROI detector means 22, a combination image formation means 80, and a hue association means 85. The image acquisition controller unit 14 further includes a focal point controller means 15 and a frequency controller means 16. The combination image formation means 80 and the hue association means 85 are identical to those shown in FIG. 2, and the detailed description thereof will be omitted. The functionality of the ROI setting means 20, the representative pixel value calculation means 21, the ROI detector means 22, the focal point controller means 15, and the frequency controller means 16 will be described in greater details later along with the operation of the controller unit 110 described below.

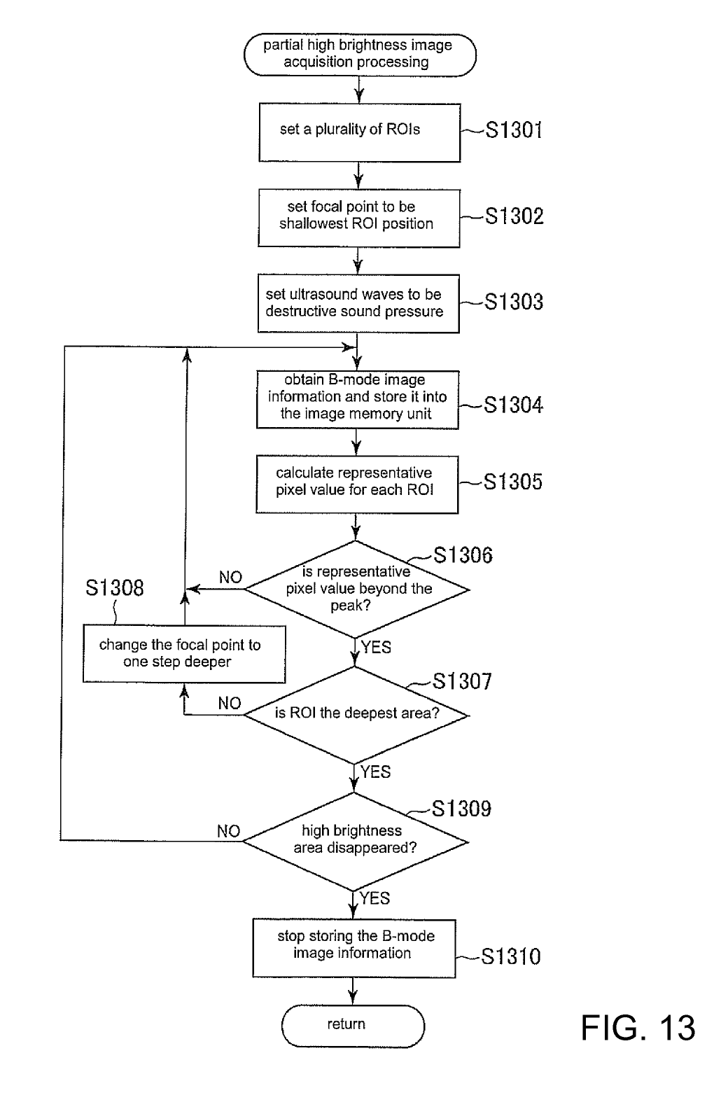

Now the operation of the controller unit 110 will be described with reference to FIG. 13. FIG. 13 shows a flow chart illustrating the operation of the partial high brightness image acquisition processing performed by the controller unit 110. The operation of the combination image formation processing performed by the controller unit 110 is identical to the operation shown in the flow chart of FIG. 3 and the detailed description thereof will be omitted.

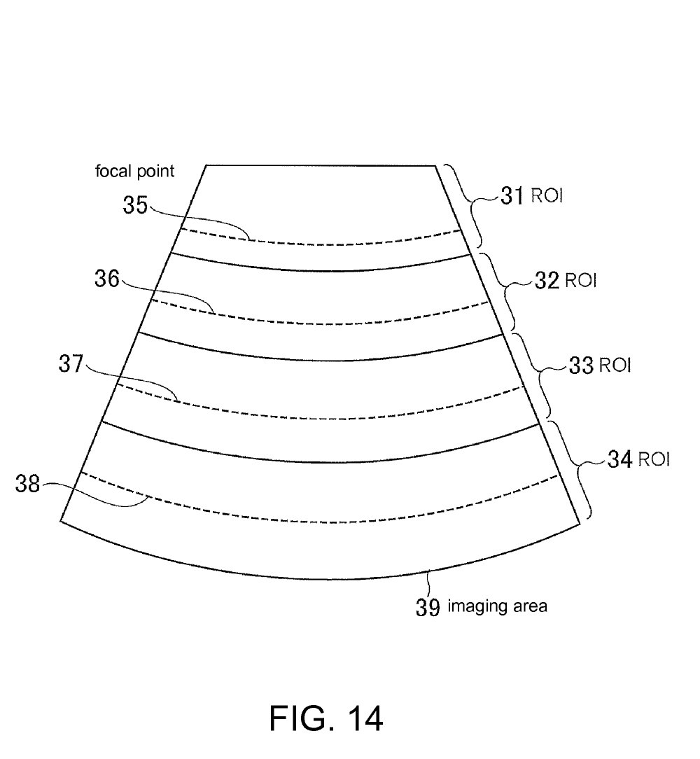

The operation instructs from the input unit 107 to set a plurality of ROIs in the depth direction of the imaging area (step S1301). FIG. 14 shows a schematic diagram illustrating an example of a plurality of ROIs set in the depth direction of the imaging area 39. The imaging area 39 indicates the imaging section within the subject 1 from which the B-mode image information is acquired, and the ROIs 31 to 34 separated apart uniformly in the depth direction are set from the input unit 107.

The ROI information set by the input unit 107 is read in to the focal point controller means 15, the frequency controller means 16, and the ROI setting means 20.

The focal point controller means 15 controls the delay circuit in the image processing unit 103 to change the focal point in the depth direction of transmitting the ultrasound waves. The focal point controller means 15 calculates the focal point based on the ROI information of the ROIs 31 to 34 set from the input unit 107. The focal point may be set for example at a position that is by the deeper side of the depth direction from the center position of the ROI in the depth direction. The focal point controller means 15 switches the focal point based on the invoke signals from the ROI detector means 22.

The frequency controller means 16 controls the driving waveform generator circuit of the transmitter/receiver unit 102 to control the frequency of the square wave pulses for exciting the piezoelectric elements. The frequency controller means 16 sets the exciting frequency optimal to these ROIs based on the information on a plurality of ROIs set by the input unit 107. For example, for a ROI that resides at a deeper position in the depth direction, the ultrasound waves of the frequency lower than for the ROI at a shallower position may be emitted so as to acquire the B-mode image information desirably having a less attenuation.

Then, the controller unit 110 sets the value of the focal point 35 that the shallowest ROI 31 has, to the transmitter/receiver unit 102 as the initial value (step S1302). The controller unit 110 also transmits to the sound pressure changing means 12 the signal that sets the sound pressure of the ultrasound waves to be the destructive sound pressure, to change the transmitted ultrasound waves to the destructive sound pressure of the contrast agent (step S1303), then the probe unit 101 is placed in contact with the target imaging area to acquire the B-mode image information, and the B-mode image information thus acquired is stored in the image memory unit 104 (step S1304).

The controller unit 110 calculates in real-time basis the representative pixel values, which represents the ROIs 31 to 34 each time the B-mode image information is stored (step S1305) then determines whether or not the representative pixel value is beyond the peak (step S1306). In this determination the ROI setting means 20 of the controller unit 110 uses the ROI information of the ROIs 31 to 34 set by the input unit 107 and the B-mode image information from the image memory unit 104 to classify and acquire the B-mode image information into ROIs 31 to 34. Then the representative pixel value calculation means 21 of the controller unit 110 uses the image information of the classified ROIS 31 to 34 to determine the representative pixel values. The representative pixel values may be the addition value, the maximum pixel value, or the mean pixel value of the pixel values of the ROIs 31 to 34 calculated for each of ROIs 31 to 34, and indicates the brightness value which represents the ROIs 31 to 34.

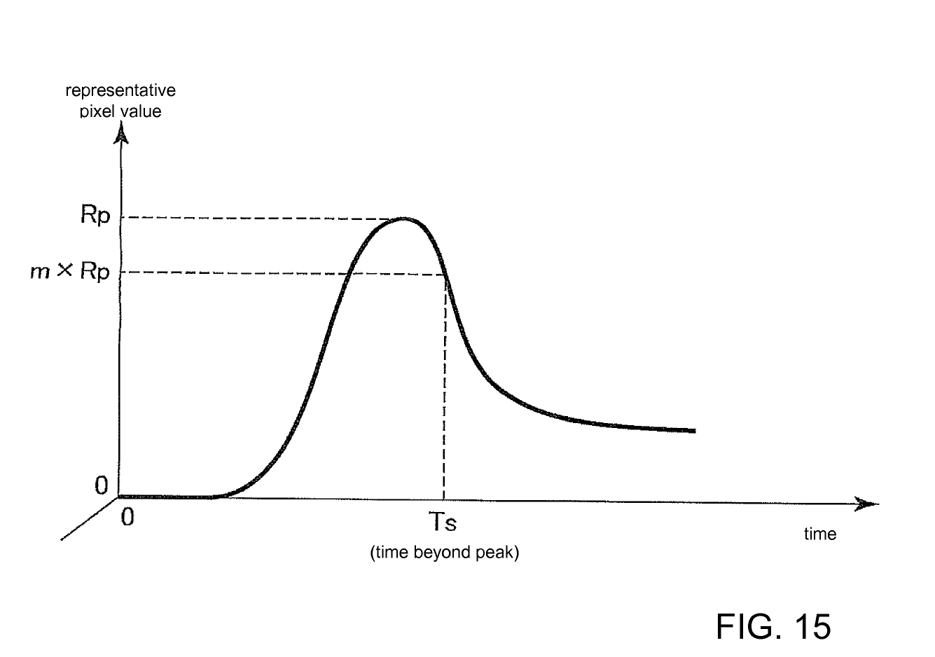

The ROT detector means 22 of the controller unit 110 obtains in real-time basis the representative pixel values for each of the ROIs 31 to 34 calculated by the representative pixel value calculation means 21 to determine the change along with the time of these representative pixel values. FIG. 15 shows a schematic diagram illustrating the change in time course of the representative pixel value of the ROI 32, such as the maximum intensity. The axis of abscissa indicates the time from starting imaging, and the axis of ordinate indicates the value of the representative pixel value. The representative pixel value of the ROI 32 indicates the change in time course as similar to the brightness change curve 82 shown in FIG. 8, and has its peak value Rp at the time approximately when the brightness of the high brightness area 46 becomes high.

The ROI detector means 22 detects in real-time basis the time Ts that the change in time course of the representative pixel value is beyond the peak. The ROI detector means 22 compares the value of the representative pixel value obtained in real-time basis with the maximum intensity predetermined, which is zero at the initial value, and sets the larger value as the new maximum intensity. At the same time the ROI detector means 22 calculates the multiplication value that is the previously set maximum intensity multiplied with a predetermined multiplication rate m, such as 0.8, and determines the time that the representative pixel value has been obtained as the time Ts that exceeds the peak, when the representative pixel value acquired in real-time basis goes below this multiplication value.

Thereafter, returning to FIG. 13, if the representative pixel value is not beyond the peak (step S1306 negative), the ROI detector means 22 proceeds to step S1304, where it repeats the acquisition of the B-mode image information and the storage into the image memory unit 104. If the representative pixel value is beyond the peak (step S1306 positive), the ROI detector means 22 determines whether the ROI resides in the deepest area (step S1307). If the ROI is not at the deepest area (step S1307 negative) then the focal point switching signal is sent to the focal point controller means 15 to change the focal point to one step deeper (step S1308).

If the ROI resides at the deepest area (step S1307 positive) then the ROI detector means 22 determines whether or not the high brightness area present in the imaging area has been disappeared in accordance with the instruction by the operator through the input unit 107 (step S1309), if the high brightness area is not disappeared from the imaging area (step S1309 negative), then the process proceeds to the step S1304, where the acquisition and the storage of the B-mode image information is repeated. If the high brightness area is disappeared from the imaging area (step S1309 positive) then the ROI detector means 22 follows the instruction from the input unit 107 by the operator to instruct to decrease the sound pressure of the transmission ultrasound waves, to stop storing the B-mode image information into the image memory unit (step S1310), then the ROI detector means 22 terminates this partial high brightness image acquisition processing, to return back to the main routine.

As have been described above, in this second embodiment, a plurality of ROIs 31 to 34, as well as the focal points 35 to 38 for each of ROIs 31 to 34 are set in the depth direction of the imaging area in the partial high brightness image acquisition processing, the ROI detector means 22 detects the ROI in which the high brightness area is present and moving toward the depth direction, and the focal point of this ROI is used as the focal point of the ultrasound waves to be transmitted, so that the high brightness area is recorded as the B-mode image information of the higher resolution, while the sound pressure is concentrated thereto, allowing forming much clearer combination image information.

In this second embodiment, although the focal point of the ultrasound waves to be emitted is set to the high brightness ROI detected by the ROI detector means 22 by using the focal point controller means 15, similarly thereto the excitation frequency of the ultrasound waves to be emitted to each of high brightness ROIs detected may be changed by using the frequency controller means 16. For example, when the high brightness ROI moves from the shallower ROI to the deeper ROI, the excitation frequency of the ultrasound waves to be emitted may be varied from a high frequency to a lower frequency to allow the B-mode image information of a higher resolution to be obtained in the shallower ROI while allowing the B-mode image information of a high sensitivity with less attenuation to be obtained in the deeper ROI.

Many widely different embodiments of the invention may be configured without departing from the spirit and the scope of the present invention. It should be understood that the present invention is not limited to the specific embodiments described in the specification, except as defined in the appended claims.

1. An ultrasound imaging apparatus comprising:
an image acquisition unit configured to emit ultrasound waves to a subject administered with a contrast agent and for obtaining a plurality of B-mode images of the subject;
an image memory unit configured to store the plurality of B-mode images, obtained by emitting ultrasound waves to the subject at a sound pressure that destroys the contrast agent; and
a combination image formation device configured to form a combination image from the plurality of B-mode images.

2. An ultrasound imaging apparatus according to claim 1, wherein the combination image is a maximum intensity projection image, said combination image formation device is configured to form the combination image by comparing pixel values at an identical pixel position of a plurality of the B-mode images, to a maximum intensity among the pixel values as the pixel value of the pixel position.

3. An ultrasound imaging apparatus according to claim 1, wherein each of the plurality of B-mode images includes B-mode image information that includes a time information indicative of a time from a start of an emission to an acquisition of each B-mode image.

4. An ultrasound imaging apparatus according to claim 3, wherein combination image formation device is configured to use the time information to form a parameter value image information in which a parameter value calculated from a temporal change of he pixel values is set as the pixel value by using the time of the start of the emission of the ultrasound waves at the destructive sound pressure as a starting time for each pixel constituting the B-mode image information.

5. An ultrasound imaging apparatus according to claim 4, wherein the parameter value is a peak pixel value that the pixel value is at the peak and a peak time that is from the start of the emission to the peak.

6. An ultrasound imaging apparatus according to claim 5, wherein the parameter value is a destruction termination time from the start of the emission to a decrease of the pixel value beyond the peak pixel value on a brightness change curve.

7. An ultrasound imaging apparatus according to claim 5, wherein the parameter value is a destruction continuation time which is a difference between the destruction termination time and the peak time.

8. An ultrasound imaging apparatus according to claim 1, further comprising a display unit configured to color display the combination image, and an image display controller unit configured to control the color display.

9. An ultrasound imaging apparatus according to claim 2, further comprising a display unit configured to color display the combination image, and an image display controller unit configured to control the color display.

10. An ultrasound imaging apparatus according to claim 3, further comprising a display unit configured to color display the combination image, and an image display controller unit configured to control the color display.

11. An ultrasound imaging apparatus according to claim 4, further comprising a display unit configured to color display the combination image, and an image display controller unit configured to control the color display.

12. An ultrasound imaging apparatus according to claim 5, further comprising a display unit configured to color display the combination image, and an image display controller unit configured to control the color display.

13. An ultrasound imaging apparatus according to claim 8, wherein said combination image formation device comprises a hue association device configured to associate a pixel value of the combination image information to a hue.

14. An ultrasound imaging apparatus according to claim 13, further comprising an image display controller unit configured to color display the combination image information associated to the hue.

15. An ultrasound imaging apparatus according to claim 1, further comprising an input unit configured to set a plurality of ROIs each having a different depth in a depth direction of the emission of ultrasound waves.

16. An ultrasound imaging apparatus according to claim 15, further comprising a representative pixel value calculation device configured to use a pixel value of each of the plurality of B-mode images for each of the plurality of ROIs to calculate one of a maximum intensity of the pixel value within each of the plurality of ROIs, an addition value of the pixel value within each of the plurality of ROIs, and an addition mean value of the pixel values within each of the plurality of ROIs as a representative pixel value.

17. An ultrasound imaging apparatus according to claim 16, further comprising a ROI detection device configured to use the representative pixel value to detect an ROI of the plurality of ROIs that has a highest brightness by the emission of the ultrasound waves of the destructive sound pressure.

18. An ultrasound imaging apparatus according to claim 17, further comprising a focal point controller device configured to change a location of a focal point of the ultrasound waves based on the detection.

19. An ultrasound imaging apparatus according to claim 17, further comprising a frequency controller device configured to change an excitation frequency of the ultrasound waves based on the detection.

20. An ultrasound imaging method comprising:
emitting ultrasound waves to a subject administered with a contrast agent;
obtaining a plurality of B-mode images of the subject;
storing the plurality of B-mode images, obtained by emitting ultrasound waves to the subject at a sound pressure that destroys the contrast agent; and
forming a combination image from the plurality of the B-mode images.
